(19) United States
(12) Patent Application Publication
(10) Pub. No.: US 2019/0374534 A1
(43) Pub. Date: Dec. 12, 2019

(54) LIQUID PHARMACEUTICAL COMPOSITIONS COMPRISING PERGOLIDE

(71) Applicant: Boehringer Ingelheim Vetmedica GmbH, Ingelheim am Rhein (DE)

(72) Inventors: Martin Andreas FOLGER, Ingelheim am Rhein (DE); Monika BRINK, Gau-Algesheim (DE); Marcus Rainer RAHMEL, Ockenheim (DE)

(21) Appl. No.: 16/431,883

(22) Filed: Jun. 5, 2019

(30) Foreign Application Priority Data
Jun. 8, 2018 (EP) 18176821.9

(51) Int. Cl. A61K 31/48; A61K 9/16; A61K 9/00; A61M 5/24; A61M 5/315; A61J 1/14; A61M 5/00

(52) U.S. Cl. CPC A61K 31/48; A61K 9/16; A61K 9/0056; A61M 5/002; A61M 5/3155; A61J 1/1412; A61K 9/1664; A61M 5/24

(57) ABSTRACT

The invention relates to novel liquid pharmaceutical compositions comprising (8β)-8-[(methylthio)methyl]-6-propylergoline (pergolide) as well as corresponding processes of manufacturing such liquid pharmaceutical compositions and their medical uses.

### RELATED APPLICATIONS

This application claims the priority of European Patent Application No. EP 18176821.9, filed 8 Jun. 2018, and is incorporated by reference in its entirety and relied upon for all purposes.

### FIELD OF THE INVENTION

The invention relates to the field of medicine, particularly veterinary medicine. In particular, the invention relates to novel liquid pharmaceutical compositions comprising pergolide.

### BACKGROUND OF THE INVENTION

Pergolide is currently available under the trade name Prascend® as tablet only, with 1 mg active pharmaceutical ingredient (API) pergolide per tablet. The tablet was developed for use in humans, is not flavored and can be divided into 2 pieces. The tablet does not provide enough flexibility for accurate dose adjustment, e.g., for small ponies. A 500 kg horse is supposed to receive 1 mg API daily.

Pertinent prior art is as follows:

U.S. Pat. No. 3,901,894 relates to 8-thiomethylergolines useful as prolactin inhibitors.

EP 0 003 667 and U.S. Pat. No. 4,166,182 describe substituted ergolines, their preparation, compositions containing them and their use as pharmaceuticals, e.g. for the inhibition of prolactin secretion or the treatment of Parkinson's syndrome.

EP 0 026 671 and U.S. Pat. No. 4,246,265 deal with D-6-n-propylergoline derivatives compositions containing them and their use as pharmaceuticals, e.g. for lowering the prolactin levels in mammals or for treating symptoms of Parkinson's syndrome in humans

EP 0 213 850 and U.S. Pat. No. 4,782,152 relates to a process for the decyanation of pergolide intermediate.

WO 96/40139 is directed to novel formulations for the transdermal delivery of pergolide.

WO 02/11727 discloses a formulation and a method of manufacturing stable pergolide mesylate.

The disadvantages of the prior art are (i) limited dose adjustment possible (0.5 mg API or 1.0 mg API), (ii) necessity of storage of half tablets representing a risk due to stability issues, (iii) no flavor is used for tablet, thus the acceptance by the animals being limited, and (iv) an administration is only into the mouth possible and cannot for instance be poured on the horse food for administration.

WO 02/15903 describes an oily suspension depot formulation of dopamine D2 agonist rotigotine (N-0923) for the treatment of Parkinson's disease and restless leg syndrome.

US 2008/260846 discloses long-acting sustained release formulations containing dopamine receptor agonists and also mentions pergolide mesylate.

Shank B et al. (Journal of Pharmacy Practice 2010, 23(6): 570-574) is directed to the evaluation of the stability of pergolide mesylate in an oral aqueous liquid.

Davis J et al. (Journal of the American Veterinary Medical Association 2009, 234(3): 385-389) relates to the evaluation of the effects of temperature and light on the stability of pergolide mesylate after compounding such in an aqueous vehicle.

There is an urgent need for a directly administrable pharmaceutical composition comprising pergolide which overcomes the problems of the prior art as described above.

### SUMMARY OF THE INVENTION

The present invention concerns a liquid pharmaceutical composition comprising (8β)-8-[(methylthio)methyl]-6-propylergoline (pergolide) according to formula (I):

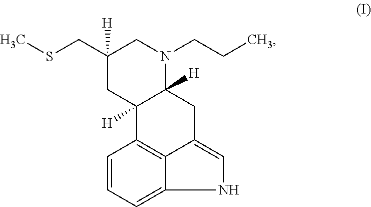 [SMILES: CCCN1C[C@H](CSC)C[C@@H]2c3cccc4[nH]cc(c34)C[C@H]21]

wherein (8β)-8-[(methylthio)methyl]-6-propylergoline [pergolide (INN)] comprises, preferably consists of, particles characterized through a particle size distribution D90 value of 300 μm or less, more preferably 250 μm or less, more preferably 200 μm or less, more preferably 150 μm or less, more preferably 100 μm or less, even more preferably 90 μm or less, even more preferably 80 μm or less, even more preferably 70 μm or less, even more preferably 60 μm or less, even more preferably 50 μm or less, more preferably 40 μm or less.

Most preferably such micronized pergolide mesylate particles are characterized through a particle size distribution D90 value of from 0.001 μm-300 μm, preferably from 0.01 μm-100 μm, more preferably from 0.1 μm-90 μm, even more preferably from 1 μm-80 μm, even more preferably from 2 μm-70 μm, even more preferably from 3 μm-60 μm, even more preferably from 4 μm-50 μm, and most preferably from 5 μm to 40 μm.

Most preferably such micronized pergolide mesylate particles are characterized through an average (mean) particle size of equal to or more than 1 μm, in particular equal to or more than 2 μm, equal to or more than 3 μm, equal to or more than 4 μm, equal to or more than 5 μm, equal to or more than 6 μm, equal to or more than 7 μm, equal to or more than 8 μm, equal to or more than 9 μm, equal to or more than 10 μm, equal to or more than 11 μm, equal to or more than 12 μm, or equal to or more than 13 μm.

Most preferably such micronized pergolide mesylate particles are characterized through a median particle size of equal to or more than 1 μm, in particular equal to or more than 2 μm, equal to or more than 3 μm, equal to or more than 4 μm, equal to or more than 5 μm, equal to or more than 6 μm, equal to or more than 7 μm, equal to or more than 8 μm, equal to or more than 9 μm, equal to or more than 10 μm, equal to or more than 11 μm, equal to or more than 12 μm, or equal to or more than 13 μm.

Most preferably such micronized pergolide mesylate particles are characterized through an average (mean) particle size of from 6 μm-65 μm, preferably from 7 μm-60 μm, more preferably from 8 μm-55 μm, even more preferably from 9 μm-50 μm, even more preferably from 10 μm-45 μm, even more preferably from 11 μm-40 μm, even more preferably from 12 μm-35 μm, and most preferably from 13 μm to 30 μm.

The present invention also concerns a liquid pharmaceutical composition as described and claimed herein for use in a method for treating and/or preventing one or more medicinal indications in a subject in need of such treatment and/or prevention, preferably an animal, more preferably a mammal, most preferably a horse, selected from among the medicinal indications: Pituitary Pars Intermedia Dysfunction (PPID; Equine Cushing's Disease).

Most preferably such micronized pergolide mesylate particles are characterized through a median particle size of from 6 μm-65 μm, preferably from 7 μm-60 μm, more preferably from 8 μm-55 μm, even more preferably from 9 μm-50 μm, even more preferably from 10 μm-45 μm, even more preferably from 11 μm-40 μm, even more preferably from 12 μm-35 μm, and most preferably from 13 μm to 30 μm.

The present invention further concerns a process for producing the liquid pharmaceutical composition as described and claimed herein, comprising the steps:
(i) pre-dispersion of pergolide in one or more oil(s) to yield a pergolide suspension;
(ii) preparation of an antioxidant solution in one or more oil(s) containing at least one antioxidant;
(iii) preparation of a suspension by mixing the pergolide suspension obtained in step (i) with the antioxidant solution obtained in step (ii);
(iv) addition of at least one flavor to the suspension obtained in step (iii);
(v) homogenization of the suspension obtained in step (iv).

The present invention further concerns a process for producing the liquid pharmaceutical composition as described and claimed herein, comprising the steps
(i) preparation of an antioxidant solution in one or more oil(s) containing at least one antioxidant;
(ii) addition of the at least one viscosity enhancer to the solution obtained in step (i) followed by homogenization;
(iii) pre-dispersion of pergolide in one or more oil(s) to yield a pergolide suspension;
(iv) preparation of a suspension by mixing the pergolide suspension obtained in step (iii) with the antioxidant+viscosity enhancer containing suspension obtained in step (ii);
(v) homogenization of the suspension obtained in step (iv);
(vi) addition of at least one flavor to the suspension obtained in step (v);
(vii) homogenization of the suspension obtained in step (vi).

The present invention further concerns a kit-of-parts comprising:
(a) a liquid pharmaceutical composition as described and claimed herein; and
(b) a package leaflet including the information that the liquid pharmaceutical composition is to be used for the prevention and/or treatment of one or more medicinal indications in a subject in need of such prevention and/or treatment, which are selected from among the medicinal indications: Pituitary Pars Intermedia Dysfunction (PPID; Equine Cushing's Disease).

In a preferred embodiment, such kit-of-parts further comprises a plasticified glass bottle for storage of the liquid pharmaceutical composition as described and claimed herein, in particular a 100 mL plasticified glass bottle, a plug-in that functions as an interface for the plasticified glass bottle and the syringe, a syringe for taking up the liquid pharmaceutical composition as described and claimed herein, preferably a syringe with dial-the-dose mechanism and six dosing steps between 0.25 mg pergolide and 1.5 mg pergolide, as well as a child-proof cap.

The present invention further concerns micronized (8β)-8-[(methylthio)methyl]-6-propylergoline (pergolide), preferably micronized pergolide mesylate, comprising, preferably consisting of, particles characterized through a particle size distribution D90 value of 300 μm or less, more preferably 250 μm or less, more preferably 200 μm or less, more preferably 150 μm or less, more preferably 100 μm or less, even more preferably 90 μm or less, even more preferably 80 μm or less, even more preferably 70 μm or less, even more preferably 60 μm or less, even more preferably 50 μm or less, even more preferably 40 μm or less.

Most preferably such micronized pergolide mesylate, comprises, preferably consists of, particles characterized through an average (mean) particle size of equal to or more than 1 μm, in particular equal to or more than 2 μm, equal to or more than 3 μm, equal to or more than 4 μm, equal to or more than 5 μm, equal to or more than 6 μm, equal to or more than 7 μm, equal to or more than 8 μm, equal to or more than 9 μm, equal to or more than 10 μm, equal to or more than 11 μm, equal to or more than 12 μm, or equal to or more than 13 μm.

Most preferably such micronized pergolide mesylate, comprises, preferably consists of, particles characterized through a median particle size of equal to or more than 1 μm, in particular equal to or more than 2 μm, equal to or more than 3 μm, equal to or more than 4 μm, equal to or more than 5 μm, equal to or more than 6 μm, equal to or more than 7 μm, equal to or more than 8 μm, equal to or more than 9 μm, equal to or more than 10 μm, equal to or more than 11 μm, equal to or more than 12 μm, or equal to or more than 13 μm.

The advantages of the liquid pharmaceutical compositions according to the present invention are as follows:
improved dosing flexibility; e.g. from 0.25 mg to 1.5 mg pergolide in 0.25 mg steps.
improved dosing accuracy;
ensurance of dosage unit through homogeneity of mixture is secured through reduced API particle size;
possibility of use of flavored suspension to increase long-term acceptability by the animals;
administration onto horse food to ease administration for horse owners.

### DETAILED DESCRIPTION OF THE INVENTION

Before the embodiments of the present invention are described in further details it shall be noted that as used herein and in the appended claims, the singular forms “a”, “an”, and “the” include plural reference unless the context clearly dictates otherwise.

Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was usually omitted from the description and claims. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention. Nothing herein is to be construed as an admission that the invention is not entitled to antedate such disclosure by virtue of prior invention.

In the course of the present invention (8β)-8-[(methylthio)methyl]-6-propylergoline (pergolide) is also referred to as “the substance”.

In the course of the present invention the term “suitable for direct administration to a subject” in connection with “liquid pharmaceutical composition” means that such liquid pharmaceutical compositions can be directly administered to a subject without further mandatory processing and/or purification steps. Preferably, such “liquid pharmaceutical composition” that are “suitable for direct administration to a subject” comply with GMP manufacturing conditions as well as GCP compliant clinical protocols.

In one aspect, the present invention relates to a liquid pharmaceutical composition as described and claimed herein, wherein the liquid pharmaceutical composition is suitable for direct administration to a subject, preferably an animal, more preferably a mammal, most preferably a horse, in particular suitable for direct administration onto horse food.

In yet another aspect, the present invention relates to a liquid pharmaceutical composition as described and claimed herein, wherein pergolide is micronized pergolide mesylate.

In yet another aspect, the present invention relates to a liquid pharmaceutical composition as described and claimed herein, wherein the liquid pharmaceutical composition is a suspension.

In yet another aspect, the present invention relates to a liquid pharmaceutical composition as described and claimed herein, wherein the suspension is an oily suspension, i.e. a suspension comprising one or more oil(s), such as mineral oil(s) and/or vegetable oil(s).

In yet another aspect, the present invention relates to a liquid pharmaceutical composition as described and claimed herein, wherein the oily suspension is devoid of any water. Preferably, the term “devoid of any water” refers to a water content of less than 3%, more preferably of less than 1%, even more preferably of less than 0.5% and most preferably of 0.0%.

In yet another aspect, the present invention relates to a liquid pharmaceutical composition as described and claimed herein, wherein the one or more oil(s) are selected form the group consisting of: refined soyabean oil, refined safflower oil, refined maize oil, virgin linseed oil, refined sunflower oil, refined rapeseed oil, and miglyol [mixture of medium-chain (e.g., C₈-C₁₀ fatty acid) triglycerides (MCT), in particular of saturated fatty acids, such as succinic acid, caprylic acid and/or capric/caprinic acid], wherein preferably the miglyol is selected from the group consisting of: Miglyol 810, Miglyol, 812, Miglyol 829, Miglyol 840, more preferably Miglyol 812.

In yet another aspect, the present invention relates to a liquid pharmaceutical composition as described and claimed herein, wherein the liquid pharmaceutical composition further comprises at least one antioxidant, preferably selected from the group consisting of: butylhydroxytoluene (BHT), ascorbyl palmitate (AP), butylhydroxyanisole (BHA), and alpha-tocopheryl acetate (AT), more preferably butylhydroxyanisole (BHA).

In yet another aspect, the present invention relates to a liquid pharmaceutical composition as described and claimed herein, wherein the liquid pharmaceutical composition does not comprise rapeseed oil and alpha-tocopheryl acetate (AT).

In yet another aspect, the present invention relates to a liquid pharmaceutical composition as described and claimed herein, wherein the liquid pharmaceutical composition further comprises at least one flavour, preferably selected from the group consisting of: apple-carrot flavour, honey-carrot flavour, more preferably honey-carrot flavour.

In yet another aspect, the present invention relates to a liquid pharmaceutical composition as described and claimed herein, wherein the liquid pharmaceutical composition further comprises at least one viscosity enhancer, preferably selected from the group consisting of: oleogel formers, such as silicium dioxide and/or ethyl cellulose, magnesium stearate, aluminium stearate, calcium stearate, zinc stearate, preferably silicium dioxide, wherein preferably the viscosity enhancer is present in a concentration of 0.1% (w/w) to 20% (w/w), more preferably 1% (w/w) to 10% (w/w), even more preferably 5% (w/w) to 10% (w/w), even more preferably 1% (w/w) to 4% (w/w), even more preferably 1% (w/w) to 2% (w/w), even more preferably 1.5% (w/w) to 2.5% (w/w), most preferably 1.5% (w/w) or 2.5% (w/w).

In yet another aspect, the present invention relates to a liquid pharmaceutical composition as described and claimed herein comprising
(i) 0.1-1.0 g/L (0.01-0.1% w/w), preferably 0.1-0.65 g/L (0.01-0.065% w/w) pergolide;
(ii) 750-995 g/L (75-99.5% w/w), preferably 850-995 g/L (85-99.5% w/w) oil;
(iii) 0.05-1 g/L (0.005-0.1% w/w), preferably 0.05-0.1 g/L (0.005-0.01% w/w) antioxidant;
(iv) 5-20 g/L (0.5-2.0% w/w), preferably 5-10 g/L (0.5-1% w/w) flavor.

In yet another aspect, the present invention relates to a liquid pharmaceutical composition as described and claimed herein comprising
(i) 0.1-1.0 g/L (0.01-0.1% w/w), preferably 0.1-0.65 g/L (0.01-0.065% w/w) pergolide;
(ii) 750-995 g/L (75-99.5% w/w), preferably 850-995 g/L (85-99.5% w/w) oil;
(iii) 0.05-1 g/L (0.005-0.1% w/w), preferably 0.05-0.1 g/L (0.005-0.01% w/w) antioxidant;
(iv) 5-20 g/L (0.5-2.0% w/w), preferably 5-10 g/L (0.5-1% w/w) flavor;
(v) 1-200 g/L (0.1-20% w/w), preferably 10-40 g/L (1-4% w/w) viscosity enhancer.

In yet another aspect, the present invention relates to a liquid pharmaceutical composition as described and claimed herein, wherein liquid pharmaceutical composition is suitable for direct administration to a subject, preferably an animal, more preferably a mammal, most preferably a horse.

In yet another aspect, the present invention relates to a liquid pharmaceutical composition as described and claimed herein, wherein such liquid pharmaceutical composition is for oral administration, preferably for administration onto horse food.

### BRIEF DESCRIPTION OF THE DRAWINGS

FIG. 1: shows the certificate of analysis for micronized pergolide mesylate with a particle size distribution D90 value of 40 μm or less (39.4 μm) and a median particle size of equal to or more than 13 μm (13.3 μm).

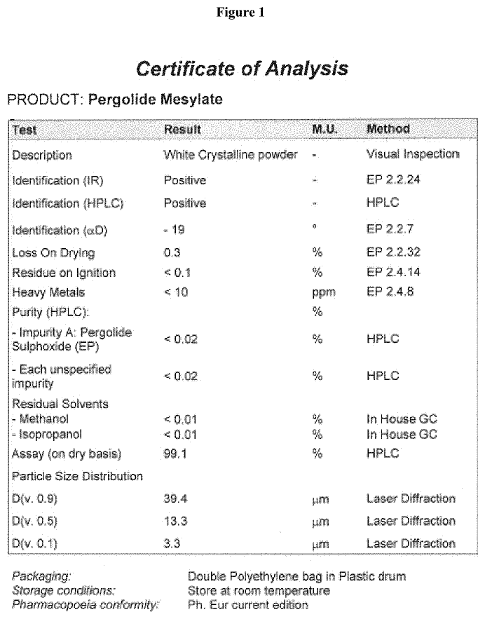

### EXAMPLES

The following examples serve to further illustrate the present invention; but the same should not be construed as a limitation of the scope of the invention disclosed herein.

### Example 1—Micronization of Pergolide Mesylate

Micronization of pergolide mesylate was performed according to the state-of-the-art using a spiral jet-mill as described below. The principles of jet-mills is for instance described in Vauck, Wilhelm R. A. “Grundoperationen chemischer Verfahrenstechnik” by Vauck, Wilhelm R. A. and Müller, Hermann A., 9^{th} edition 1992, pages 329 to 332.

EQUIPMENT USED FOR MICRONIZATION: Glove box on a MC50 spiral jet-mill with a bottom discharge of the product and using nitrogen as micronization and inertization gas.

PARTICLE-SIZE DISTRIBUTION (PSD) ANALYTICAL METHOD: Equipment Malvern Mastersizer 3000 equipped with Hydro MV dispersion unit

Dispersant Preparation—1. Fill the dispersion unit with hexane—2. Check that the channel 1 of background signal is less than 80 and no anomalous spikes are present—3. Check the background signal: it has to be stable and complying with the following limits: Channel 1<100, Channel 20<60.

Sample Preparation—Randomize the sample by manually tumbling and rolling for 30 seconds, weigh approximately 15 mg of powder in a 100 mL Erlenmeyer flask, add 2 mL of Span 85 (Sorbitane trioleate; 5% w/w water solution). A homogeneous mixture, using the tip of a small stainless steel spatula, has to be obtained.

Using a Pasteur pipette add 1 mL of water and homogenize the suspension. Repeat this operation until a volume of 8-10 mL is reached. Sonicate the sample for 5 seconds in an ultrasonic bath (25 kHz).

Parameters:

Optical Model—Fraunhofer

Background measurement duration (red)—30

Sample measurement duration (red)—30

Number of measurements—3

Averaging enabled?—No

Pre-alignment delay—0

Delay between measurements—0

Pre-measurement delay—30.00 s

Auto start measurement—no

Obscuration low limit—15%

Obscuration high limit—25%

Background Check Limits—[1:100]; [20;60]

Stirrer speed—3000 rpm

Ultrasonication duration—0.00 s

Analysis model—general purpose

Analysis sensitivity—enhanced

Procedure—1. Once a stable background signal is obtained, proceed with the SOP—2. Add the sample until the obscuration target is reached. Avoid droplets to fall directly into the liquid when adding the sample suspension putting the pipette tip under water—3. Initiate the measurement—4. The particle size distribution values (D10, D50 and D90) are taken directly from the Malvern screen. All three values determined for a sample are average and rounded to obtain the final values.

### Example 2—Preparation of a Liquid Pharmaceutical Composition According to the Present Invention

Without Nitrogen Purge:

The oily suspension is manufactured in an ointment processing vessel, equipped with stirrer, homogenizer, heating-/cooling jacket and vacuum pump, e.g. a Becomix, type Beco mini Lab.

In a first step, the antioxidant (0.010%/0.340 g) is dissolved separately utilizing sufficient volumes (5%/170 g) of Miglyol 812.

Second, the micronized API pergolide mesylate (0.069%/2.337 g; D90=39.4 μm) is predispersed rapidly in a low amount of Miglyol 812 (5%/170 g) and added to the processing vessel. The micronized pergolide mesylate gets dispersed in the Miglyol-antioxidant-solution while stirring (5-10 min) and homogenization (rotor/stator principle, 8-12 m/s). If temperature increases above 25° C., the cooling jacket is turned on (below 25° C.). Honey carrot- or/and apple-carrot flavor is added (1%/34 g), followed by a homogenization step (rotor/stator principle, 8-12 m/s, 2-4 min). During the whole process, suitable process parameters (stirring time and speed, homogenization time and speed) are mandatory. The final suspension gets discharged via the homogenization element and filled into bottles and stored in a dark place.

With Nitrogen Purge:

The oily suspension is manufactured in an ointment processing vessel, equipped with stirrer, homogenizer, heating-/cooling jacket and vacuum pump, e.g. a Becomix, type Beco mini Lab. The process is run in nitrogen atmosphere.

In a first step, the antioxidant (0.010%/0.340 g) is dissolved separately utilizing sufficient volumes (5%/170 g) of Miglyol 812.

Second, the micronized API pergolide mesylate (0.069%/2.337 g; D90=39.4 μm) is predispersed rapidly in a low amount of Miglyol 812 (5%/170 g) and added to the processing vessel. The micronized pergolide mesylate gets dispersed in the Miglyol-antioxidant-solution while stirring (5-10 min) and homogenization (rotor/stator principle, 8-12 m/s). If temperature increases above 25° C., the cooling jacket is turned on (below 25° C.). Honey carrot- or/and apple-carrot flavor is added (1%/34 g), followed by a homogenization step (rotor/stator principle, 8-12 m/s, 2-4 min). During the whole process, suitable process parameters (stirring time and speed, homogenization time and speed) are mandatory The final suspension gets discharged via the homogenization element and filled into bottles. The bottles are flooded with nitrogen and stored in a dark place.

Preferred parameter setup: API adding: Homogenizer speed 8 m/s; API adding: Homogenizer time: 5 min; API adding: Stirring speed: 1.0 m/s; Flavour adding: Homogenizer speed 8 m/s; Flavour adding: Homogenizer time: 4 min; Flavour adding: Stirring speed: 1.0 m/s

### Example 3—Preparation of a Liquid Pharmaceutical Composition According to the Present Invention

The antioxidant (butyl hydroxyl anisole; 0.34 g) is dissolved in Miglyol 812 (170.0 g) while stirring until the solution is clear and free of undissolved particles. 2,717.3 g Miglyol 812 are given into a Becomix; and the antioxidant containing solution is added slowly while stirring. The beaker of the antioxidant containing solution is rinsed with 85.0 g Miglyol 812 and given into the Becomix as well. The becomix is closed and flooded with nitrogen while stirring. The at least one viscosity enhancer (Aerosil 200 Pharma; 51.00 g or 85.00 g) is slowly given to the solution while stirring and homogenizing. The beaker of the resulting suspension is rinsed with 85.0 g Miglyol 812 and given into the Becomix as well. The becomix is closed and flooded with nitrogen while stirring until the suspension is clear and free of bigger lumps. Pergolide mesylate (2.3358 g) is dispersed in 170.0 g Miglyol 812 until it is completely dispersed and the dispersion is free of bigger lumps. Since pergolide mesylate is sensitive to oxygen, the contact to air should be avoided as much as possible. The resulting dispersion is added to the at least one viscosity enhancer containing suspension while stirring. The beaker of the dispersion is rinsed with 85.0 g Miglyol 812 and given into the Becomix as well. The becomix is closed and flooded with nitrogen while stirring until the suspension is clear and free of bigger lumps. The resulting combined suspension is homogenized until it is free of lumps. 34.0 g apple-carrot flavor are added slowly while stirring and given into the Becomix as well. The becomix is closed and flooded with nitrogen. The resulting final suspension is homogenized until it is homogenous and free of lumps. The suspension is filled immediately into bottles while stirring and homogenizing. The bottles are flooded with nitrogen and stored in a dark place.

### REFERENCES

(1) Davis J et al., Journal of the American Veterinary Medical Association 2009, 234(3): 385-389
(2) EP 0 003 667
(3) EP 0 026 671
(4) EP 0 213 850
(5) Shank B et al., Journal of Pharmacy Practice 2010, 23(6): 570-574
(6) U.S. Pat. No. 3,901,894
(7) U.S. Pat. No. 4,166,182
(8) U.S. Pat. No. 4,246,265
(9) U.S. Pat. No. 4,782,152
(10) US 2008/260846
(11) Vauck, Wilhelm R. A. “Grundoperationen chemischer Verfahrenstechnik” by Vauck, Wilhelm R. A. and Müller, Hermann A., 9^{th} edition 1992, pages 329 to 332.
(12) WO 96/40139
(13) WO 02/11727

1. A liquid pharmaceutical composition comprising (8β)-8-[(methylthio)methyl]-6-propylergoline (pergolide) according to formula (I): 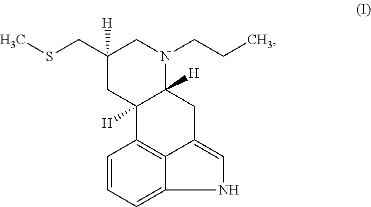 [SMILES: CCCN1C[C@H](CSC)C[C@@H]2c3cccc4[nH]cc(c34)C[C@H]21]
wherein (8β)-8-[(methylthio)methyl]-6-propylergoline (pergolide) comprises, preferably consists of, particles characterized through a particle size distribution D90 value of 300 μm or less, more preferably 250 μm or less, more preferably 200 μm or less, more preferably 150 μm or less, more preferably 100 μm or less, even more preferably 90 μm or less, even more preferably 80 μm or less, even more preferably 70 μm or less, even more preferably 60 μm or less, even more preferably 50 μm or less, most preferably 40 μm or less.

2. The liquid pharmaceutical composition according to claim 1, wherein pergolide is micronized pergolide mesylate.

3. The liquid pharmaceutical composition according to claim 1, wherein the liquid pharmaceutical composition is a suspension.

4. The liquid pharmaceutical composition according to claim 3, wherein the suspension is an oily suspension, i.e. a suspension comprising one or more oil(s), such as mineral oil(s) and/or vegetable oil(s).

5. The liquid pharmaceutical composition according to claim 4, wherein the oily suspension is devoid of any water.

6. The liquid pharmaceutical composition according to claim 4, wherein the one or more oil(s) are selected form the group consisting of: refined soyabean oil, refined safflower oil, refined maize oil, virgin linseed oil, refined sunflower oil, refined rapeseed oil, and miglyol [mixture of medium-chain triglycerides (MCT), in particular of saturated fatty acids, such as caprylic acid and capric/caprinic acid], wherein preferably the miglyol is selected from the group consisting of: Miglyol 810, Miglyol, 812, Miglyol 829, Miglyol 840, more preferably Miglyol 812.

7. The liquid pharmaceutical composition according to claim 1, wherein the liquid pharmaceutical composition further comprises at least one antioxidant, preferably selected from the group consisting of: butylhydroxytoluene (BHT), ascorbyl palmitate (AP), butylhydroxyanisole (BHA), and alpha-tocopheryl acetate (AT), more preferably butylhydroxyanisole (BHA).

8. The liquid pharmaceutical composition according to claim 4, wherein the liquid pharmaceutical composition is substantially free of rapeseed oil and alpha-tocopheryl acetate (AT).

9. The liquid pharmaceutical composition according to claim 1, wherein the liquid pharmaceutical composition further comprises at least one flavour, preferably selected from the group consisting of: apple-carrot flavour, honey-carrot flavour, more preferably honey-carrot flavour.

10. The liquid pharmaceutical composition according to claim 1, wherein the liquid pharmaceutical composition further comprises at least one viscosity enhancer, preferably selected from the group consisting of: oleogel formers, such as silicium dioxide and/or ethyl cellulose, magnesium stearate, aluminium stearate, calcium stearate, zinc stearate, preferably silicium dioxide, wherein preferably the viscosity enhancer is present in a concentration of 0.1% (w/w) to 20% (w/w), more preferably 1% (w/w) to 10% (w/w), even more preferably 5% (w/w) to 10% (w/w), even more preferably 1% (w/w) to 4% (w/w), even more preferably 1% (w/w) to 2% (w/w), even more preferably 1.5% (w/w) to 2.5% (w/w), most preferably 1.5% (w/w) or 2.5% (w/w).

11. The liquid pharmaceutical composition according to claim 1, comprising
(i) 0.1-1.0 g/L (0.01-0.1% w/w), preferably 0.1-0.65 g/L (0.01-0.065% w/w) pergolide;
(ii) 750-995 g/L (75-99.5% w/w), preferably 850-995 g/L (85-99.5% w/w) oil;
(iii) 0.05-1 g/L (0.005-0.1% w/w), preferably 0.05-0.1 g/L (0.005-0.01% w/w) antioxidant;
(iv) 5-20 g/L (0.5-2.0% w/w), preferably 5-10 g/L (0.5-1% w/w) flavor.

12. The liquid pharmaceutical composition according to claim 10 further comprising
(v) 1-200 g/L (0.1-20% w/w), preferably 10-40 g/L (1-4% w/w) viscosity enhancer.

13. The liquid pharmaceutical composition according to claim 1, wherein the liquid pharmaceutical composition is suitable for direct administration to a subject, preferably an animal, more preferably a mammal, most preferably a horse.

14. The liquid pharmaceutical composition according to claim 1, wherein such liquid pharmaceutical composition is for oral administration, preferably for administration onto horse food.

15. Method for treating and/or preventing one or more medicinal indications in a subject in need of such treatment and/or prevention, preferably an animal, more preferably a mammal, most preferably a horse, selected from among the medicinal indications: Pituitary Pars Intermedia Dysfunction (PPID; Equine Cushing's Disease), comprising administering the liquid pharmaceutical composition according to claim 1.

16. A process for producing the liquid pharmaceutical composition according to claim 1 comprising the steps:
(i) dispersing pergolide in one or more oil(s) to yield a pergolide suspension;
(ii) preparing an antioxidant solution in one or more oil(s) containing at least one antioxidant;
(iii) preparing a second suspension by mixing the pergolide suspension obtained in step (i) with the antioxidant solution obtained in step (ii);
(iv) adding at least one flavor to the second suspension obtained in step (iii); and
(v) homogenizing the suspension obtained in step (iv).

17. A process for producing the liquid pharmaceutical composition according to claim 1 comprising the steps:
(i) preparing an antioxidant solution in one or more oil(s) containing at least one antioxidant;
(ii) adding the at least one viscosity enhancer to the solution obtained in step (i) followed by homogenization;
(iii) dispersing pergolide in one or more oil(s) to yield a pergolide suspension;
(iv) preparing a suspension by mixing the pergolide suspension obtained in step (iii) with the antioxidant+viscosity enhancer containing suspension obtained in step (ii);
(v) homogenizing the suspension obtained in step (iv);
(vi) adding at least one flavor to the suspension obtained in step (v); and
(vii) homogenizing the suspension obtained in step (vi).

18. A kit comprising:
(a) a liquid pharmaceutical composition according to claim 1 and
(b) a package leaflet including the information that the liquid pharmaceutical composition is to be used for the prevention and/or treatment of one or more medicinal indications in a subject in need of such prevention and/or treatment, which are selected from among the medicinal indications: Pituitary Pars Intermedia Dysfunction (PPID; Equine Cushing's Disease),
wherein preferably such kit-of-parts further comprises a plasticified glass bottle for storage of the liquid pharmaceutical composition.

19. The kit according to claim 18 wherein the plasticified glass bottle is a 100 mL plasticified glass bottle.

20. The kit according to claim 19 further comprising a plug-in that functions as an interface for the plasticified glass bottle and a syringe sufficient to deliver the liquid pharmaceutical composition.

21. The kit of claim 20 wherein the syringe is a dial-a-dose-type syringe.

22. The kit of claim 21 wherein the dial-a-dose-type syringe comprises six dosing steps between 0.25 mg pergolide and 1.5 mg pergolide.

23. The kit of claim 18 wherein the plasticified glass bottle comprises a child-proof cap.
